(19) United States
(12) Patent Application Publication
(10) Pub. No.: US 2026/0318005 A1
(43) Pub. Date: Oct. 15, 2026

(54) Dispersible Moist Wipe Containing Natural Non-Wood Fibers

(71) Applicant: Kimberly-Clark Worldwide, Inc., Neenah, WI (US)

(72) Inventors: Thomas G. Shannon, Neenah, WI (US); David A. Moline, Appleton, WI (US); Bo Shi, Neenah, WI (US)

(21) Appl. No.: 19/484,110

(22) PCT Filed: May 31, 2024
(86) PCT No.: PCT/US2024/031968

§ 371 Date: Nov. 13, 2025

Related U.S. Application Data
(60) Provisional application No. 63/505,134, filed on May 31, 2023.

(51) Int. Cl. D21H 11/12; D04H 1/425; D04H 1/4258; D04H 1/4382; D04H 1/492; D21H 13/08; D21H 27/00

(52) U.S. Cl. CPC D21H 11/12; D04H 1/425; D04H 1/4258; D04H 1/43835; D04H 1/492; D21H 13/08; D21H 27/005; D10B 2401/063; D10B 2509/026

(57) ABSTRACT

The present disclosure is generally directed to moist or pre-moistened wipes with high wet tensile strength in combination with a low dispersibility time. The dispersible moist wipes are primarily made from non-wood pulp fibers, particularly Hesperaloe fibers.

### CROSS-REFERENCE TO RELATED APPLICATION

The present application is related and has right of priority to U.S. Provisional Patent Application No. 63/505,134 filed on May 31, 2023, which is incorporated by reference in its entireties for all purposes.

### BACKGROUND

Dispersible moist wipes are generally intended to be used and then flushed down a toilet. Accordingly, it is desirable for such flushable moist wipes to have an in-use strength sufficient to withstand a user's extraction of the wipe from a dispenser and the user's wiping activity, but then relatively quickly breakdown and disperse in household and municipal sanitization systems, such as sewer or septic systems. Some municipalities may define “flushable” through various regulations. In the relatively recent past, even stricter guidelines have been proposed and/or established that require rapid dispersibility of flushable wipes. Flushable moist wipes should meet these regulations to allow for compatibility with home plumbing fixtures and drain lines, as well as the disposal of the product in onsite and municipal wastewater treatment systems.

One well known challenge for constructing dispersible moist wipes is finding a balance between dispersibility of the wipe sufficient to meet all government regulations while still having sufficient wet tensile strength. In particular, the dispersible moist wipe should have sufficient wet tensile strength in order to perform in use yet be sufficiently weak so as to disperse under mild agitation as the product works its way through the toilet and sanitary sewer system.

In the past, flushable moist wipes were formed by hydroentangling fibers in a nonwoven web. The nonwoven web contained wood pulp fibers, particularly Northern softwood kraft fibers. In order to achieve dispersibility, the softwood fibers were combined with staple fibers, such as synthetic staple fibers or regenerated cellulose staple fibers. Synthetic fibers, however, often do not readily disperse when flushed down a toilet and do not biodegrade.

Regenerated cellulose staple fibers having relatively long fiber lengths and in relatively great amounts can produce dispersible wipes when combined with softwood fibers. The moist wipes, however, can have marginal wet strength characteristics. In addition, the use of regenerated cellulose staple fibers can add significant cost to the product as well as to the environment.

Consequently, a need currently exists for a dispersible moist wipe that not only rapidly disperses in water but also has sufficient wet tensile strength. A need also exists for a dispersible moist wipe that minimizes the amount of staple fibers present in the product in order to reduce cost and increase sustainability.

### SUMMARY

In general, the present disclosure is directed to dispersible moist wipes that contain non-wood fibers that have been found to produce products that are not only highly dispersible in water but also have excellent wet strength characteristics. The non-wood fibers, for instance, can be obtained from plants of the Hesperaloe genus. The fibers, once pulped, can have a relatively low coarseness and a relatively long natural length. The pulped Hesperaloe fibers can be optionally combined with staple fibers, such as relatively low amounts of regenerated cellulose fibers and subjected to a hydroentangling process for producing a base sheet that is then combined with a wetting solution for producing the moist wipe.

For example, in one embodiment, the present disclosure is directed to a dispersible moist wipe comprising a base sheet containing non-wood pulp fibers in an amount of greater than 50% by weight. The non-wood pulp fibers can have a coarseness of less than about 10 mg/100 m (1.0 dTex) and can have an average fiber length of from about 1.5 mm to about 3 mm. A wetting solution can be incorporated into the base sheet. In accordance with the present disclosure, the moist wipe can have a wet geometric mean tensile strength of from about 250 g/in to about 1400 g/in and a dispersibility time in seconds according to the following relationship:
1.3× wet geometric mean tensile strength (seconds).

For example, the moist wipe can have a wet geometric mean tensile strength of greater than about 300 g/in, such as greater than about 550 g/in. The moist wipe can display a dispersibility time of less than about 500 seconds, such as less than about 400 seconds, such as less than about 350 seconds, such as less than about 300 seconds, such as less than about 250 seconds, such as less than about 200 seconds, such as even less than about 180 seconds.

As described above, the non-wood fibers can be derived from one or more plants of the genus Hesperaloe. Particular Hesperaloe plants include Hesperaloe funifera, Hesperaloe parviflora, Hesperaloe noctuma, Hesperaloe chiangic, Hesperaloe tenuifolia, Hesperaloe engelmannii, Hesperaloe malacophylla, or mixtures thereof. In one aspect, the pulped non-wood fibers comprise Hesperaloe funifera fibers. The non-wood fibers can also be bleached. The non-wood fibers can be present in the base sheet in an amount greater than about 60% by weight, such as in an amount greater than about 70% by weight, such as in an amount greater than about 80% by weight, such as in an amount greater than about 90% by weight, such as even in an amount greater than about 95% by weight. The non-wood fibers can display a freeness of from about 400 mL to about 600 mL. As described above, the base sheet can also optionally contain staple fibers, such as regenerated cellulose staple fibers. The staple fibers can have an average fiber length of from about 4 mm to about 14 mm, such as from about 5 mm to about 11 mm. The staple fibers can have a decitex of from about 0.7 g/10,000 m to about 2 g/10,000 m.

In one aspect, the base sheet can be foam formed. In addition, the base sheet can be hydroentangled. The base sheet can comprise, in one embodiment, a single ply web that is non-layered. The base sheet can have a basis weight of greater than about 50 gsm, such as greater than about 60 gsm, such as greater than about 70 gsm, and less than about 150 gsm, such as less than about 130 gsm, such as less than about 100 gsm, such as less than about 90 gsm. The wetting solution can be incorporated into the base sheet in an amount that increases the weight of the base sheet from about 100% to about 400%.

The present disclosure is also directed to a wiping product that comprises a plurality of individual sheets of the dispersible moist wipes as described above. The plurality of individual sheets can have a stacked arrangement.

The present disclosure is also directed to a method of producing a dispersible moist wipe. The method includes forming a fibrous suspension. The fibrous suspension contains non-wood pulp fibers in an amount of greater than 50% by weight based on the weight of fibers present. The non-wood pulp fibers may have a fiber coarseness less than about 10.0 mg/100 m (1.0 dTex), such as less than about 8.0 mg/100 m (0.8 dTex), such as less than about 6.0 mg/100 m (0.6 dTex), such as from about 3.0 to about 10.0 mg/100 m, such as from about 3.5 mg/100 m to about 7.0 mg/100 m. The suspension of fibers optionally contains staple fibers, such as regenerated cellulose fibers. The fibers are suspended within a foam.

The fibrous suspension is deposited onto a forming surface to form a base sheet. The base sheet is subjected to a plurality of liquid jets in order to hydroentangle the fibers. The base sheet can then be dried and a wetting solution can be incorporated into the base sheet for forming a dispersible moist wipe. The moist wipe can have a wet geometric mean tensile (GMT) strength of from about 250 g/in to about 1,400 g/in and a dispersibility time in seconds of less than 1.3× the wet geometric mean tensile strength of the moist wipe. In one aspect, the base sheet contains Hesperaloe funifera pulp fibers in an amount of greater than about 70% by weight.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a diagram of one embodiment of a process for producing base sheets in accordance with the present disclosure;

FIG. 2 is a diagram of an enlarged portion of the process illustrated in FIG. 1; and

FIG. 3 is a graphical representation of the results described in the example below.

Repeat use of reference characters in the present specification and drawings is intended to represent the same or analogous features or elements of the present invention.

### Definitions

The term “machine direction” as used herein refers to the direction of travel of the forming surface onto which fibers are deposited during formation of a nonwoven web.

The term “cross-machine direction” as used herein refers to the direction which is perpendicular to the machine direction defined above.

As used herein, the term “nonwoven web or material” refers to a web having a structure of individual fibers that are interlaid, but not in an identifiable manner as in a knitted or woven fabric. Nonwoven materials include, for example, carded webs, wet-laid webs, airlaid webs, foam-formed webs, and the like.

As used herein, the term “Biomass” generally refers to organic matter derived from a non-woody plant and includes both whole plants and plant organs (i.e., leaves, stems, flowers, roots, etc.). As used herein, the term “Bagasse” generally refers to biomass that has been subjected to a processing step such as, for example, pressing, milling, compression, or maceration, to remove a portion of the biomass water soluble solids. In certain embodiments, bagasse is prepared by subjecting the biomass to compression and maceration using a plug screw, or other form of compression screw, to extract a portion of the biomass water soluble solids.

As used herein, the term “Pulp” generally refers to a plurality of cellulose fibers that have undergone a pulping process such that the fibers have become individualized and have an elongate shape in which the apparent length exceeds the apparent width. Pulp fibers can be fibrillated and can have a measurable freeness.

As used herein, the term “Fines” generally refers to fibrous water insoluble cellulosic material having a length to width aspect ratio of from about 1 to about 100 and wherein the length of the fibrous water insoluble material is less than about 0.2 mm. In certain embodiments, the amount of fines present in pulp prepared according to the present invention may be about 5.0% or less, such as about 2% or less, such as about 1.0% or less, such as from about 0.5 to about 2.0%. The fines content of pulp, on a length weighted basis, may be measured using an OpTest Fiber Quality Analyzer-360 (Op Test Equipment, Inc., Hawkesbury, ON) as described in the Test Methods section below. Generally, the percentage of fines on a length weighted basis is the sum of the fines length divided by the total length of fibers and fines in the sample.

As used herein, the term “Brightness” generally refers to the optical brightness of a pulp sample measured in accordance with ISO 2470-1:2016. Brightness is commonly expressed as a percentage (%).

As used herein, the term “Debris” generally refers to the weight percentage of solids retained on a MasterScreen™ apparatus fitted with a screen having a slot size of 100 μm (0.004 inches). The amount of debris in a given pulp sample is generally measured as set forth in the Test Methods section below.

As used herein, the term “Caliper” is the representative thickness of a pulp sheet and is generally measured as described in the Test Methods section below. Caliper commonly has units of millimeters or microns.

As used herein, the term “Freeness” refers to the Canadian Standard Freeness (CSF) determined in accordance with TAPPI Standard T 227 OM-94. Freeness commonly has units of milliliters (mL).

As used herein, the term “Fiber Length” generally refers to the length weighted average fiber length (LWAFL) of fibers measured using an OpTest Fiber Quality Analyzer, model FQA-360 (OpTest Equipment, Inc., Hawkesbury, ON) as described in the Test Methods section below. Fiber length commonly has units of millimeters.

As used herein, the term “Coarseness” generally refers to the weight per unit length of fiber measured using an OpTest Fiber Quality Analyzer-360 (OpTest Equipment, Inc., Hawkesbury, ON) as described in the Test Methods section below. Coarseness commonly has units of mass per unit length, such as milligrams per 100 meters (mg/100 meters).

As used herein, the term “Hesperaloe Fiber” refers to a fiber derived from a plant of the genus Hesperaloe of the family Asparagaceae including, for example, H. funifera, H. parviflora, H. nocturna, H. chiangii, H. tenuifolia, H. engelmannii, and H. malacophylla. The fibers are generally processed into a pulp for use in the manufacture of tissue products according to the present invention. Preferably the pulping process is a high yield pulping process, such as a pulping process having a yield greater than about 60 percent, such as from about 60 to about 90 percent and more preferably from about 65 to about 90 percent. The foregoing yields generally refer to the yield of unbleached hesperaloe fiber.

As used herein, the terms “Geometric Mean Tensile” (GMT) refer to the square root of the product of the machine direction tensile strength and the cross-machine direction tensile strength of the web.

As used herein the term “Synthetic Fiber” means a non-cellulosic, thermoplastic fiber.

As used herein the term “Thermoplastic” means a plastic which becomes pliable or moldable above a specific temperature and returns to a solid state upon cooling. Exemplary thermoplastic fibers suitable for the present embodiments include polyesters (e.g., polyalkylene terephthalates such as polyethylene terephthalate (PET), polybutylene terephthalate (PBT) and the like), polyalkylenes (e.g., polyethylenes, polypropylenes and the like), poyacrylonitriles (PAN), and polyamides (nylons, for example, nylon-6, nylon 6,6, nylon-6,12, and the like). Preferred are PET fibers.

As used herein, the term “substantially free” means less than 3 wt %, alternatively less than 2 wt %, alternatively less than 1 wt %, alternatively less than 0.5 wt %, alternatively less than 0.25 wt %, alternatively less than 0.1 wt %, alternatively less than 0.05 wt %, alternatively less than 0.01 wt %, and/or alternatively free of. As used herein, “free of” means 0 wt %.

### Test Methods

### Fiber Properties

Fiber properties such as length, coarseness, percentage of fines, and fraction of very long fiber, are generally determined using an Op Test Fiber Quality Analyzer-360 (OpTest Equipment, Inc., Hawkesbury, ON) in accordance with the manufacturer's instructions. Samples are generally prepared by first accurately weighing a pulp sample. The sample mass may range from about 10 to about 50 mg (bone dry) and may be taken from a handsheet or pulp sheet. The weighed sample is diluted to a known consistency (between about 2 and about 10 mg/l). An aliquot of the diluted sample (usually 200 ml) is further diluted to a final volume of 600 ml and placed in the analyzer. The sample is then analyzed according to the manufacturer's instructions and the output of the analyzer, such as the length weighted average fiber length, coarseness, length weighted fines, and a histogram illustrating the distribution of various fiber properties for a given sample are recorded. Generally, each reported fiber property is the average of three replicates.

The output of the fiber quality analyzer is used to calculate the Very Long Fiber (VLF) fraction, which is the sum of fiber count from 6 to 14.95 mm divided by the total fiber count. Generally, the bin data output by the instrument, which provides the number of individual fibers counted within a given fiber length range, is used to determine VLF. The total number of individual fibers counted (N) and the total number of individual fibers counted having a length of 6 mm or greater (n) are determined from the bin data. The % VLF=n/N*100.

The output of the fiber quality analyzer is also used to calculate the ratio of the length weighted average fiber length (L_{w}) to the number average fiber length (Lₙ). L_{w} and Lₙ are calculated by the FQA software using the following equations:

$${L}_{w} = \frac{{\sum }_{All Fibers} {n}_{\text{?}} {{L}_{\text{?}}}^{2}}{{\sum }_{All \text{?}} {n}_{\text{?}} {L}_{\text{?}}}$$

$${L}_{n} = \frac{{\sum }_{All Fibers} {n}_{\text{?}} {L}_{\text{?}}}{{\sum }_{All \text{?}} {n}_{\text{?}}}$$

$$\text{?} \text{indicates text missing or illegible when filed}$$

Where n and L are determined by the instrument in the course of analyzing a sample. The ratio of the length weighted average fiber length (L_{w}) to the number average fiber length (Lₙ) indicates the fiber length distribution of the sample. A higher ratio is indicative of a broader fiber length distribution. A value of 1 indicates that all of the fibers in the sample have the same length.

Fiber coarseness is measured using the FQA instrument and is measured “as-is” without removal of fines. Consistency of the pulp sample is determined using TAPPI methods T-240 or the equivalent and the consistency (%) is recorded to the nearest 0.01%. Based upon the measured consistency, the amount of undried sample required to yield approximately 0.015 grams of oven dried pulp is calculated and weighed out and the weight recorded to the nearest 0.0001 g. The weighed undried pulp is transferred to a British pulp disintegrator or equivalent pulp disintegrator and the total volume of the sample is diluted to 2 liters with deionized water and disintegrated 15,000 revolutions according to the manufacturer's instructions. The disintegrated sample is further diluted with deionized water to a total volume of 5 liters±50 mL and the volume is recorded to the nearest 10 mL. The diluted sample is agitated by stirring and approximately 600 grams are weighted out into a clean beaker. The mass of the sample weighed out to the beaker is recorded to the nearest 0.1 g. The oven dried weight of the pulp sample to be analyzed is then calculated as shown in the equation below, and fiber analysis is carried out according to the manufacturer's instructions.

$$O . D . Mass of Pulp ( g ) = \frac{\text{?}}{\text{?}}$$

$$\text{?} \text{indicates text missing or illegible when filed}$$

### Caliper

Generally, sheets are dried and prepared for testing as set forth in TAPPI T 205 sp-02. Pulp sheets may be tested as is. Caliper is measured using an L & W Model code SE 050 Micrometer or equivalent. The micrometer has a circular pressure foot having an area of 2.0 cm², a lowering speed of 1.0 mm/second and a pressure of 50 kPa. Generally, caliper is reported as the average of five samples.

### Basis Weight

Generally, sheets are dried and prepared for testing as set forth in TAPPI T 205 sp-02. Pulp sheets may be tested as is. The bone dry basis weight is generally measured by first cutting the samples to a specimen size of approximately 19.05×19.05 cm using an appropriate cutting tool. The cut sample is then placed on a balance in an oven preheated to 105±2° C. Once the weight of the sample has stabilized, the weight is recorded to the nearest 0.01 gram. The bone dry basis weight equals the measured weight (W) multiplied by 27.56.

### Tensile Strength

Generally Tensile is measured by forming a sheet of a particular pulp, as described herein, and then testing the resulting sheet. Generally, sheets are dried and prepared for testing as set forth in TAPPI T 205 sp-02. Samples are preconditioned and tested under TAPPI conditions (50±2% relative humidity and 72±1.8° F.) as set forth in TAPPI T 402. Tensile testing is carried out substantially as described in TAPPI T 494 om-01 using an MTS Systems Sintech 11S, Serial No. 6233 tensile testing instrument. The data acquisition software was an MTS TestWorks® for Windows Ver. 3.10 (MTS Systems Corp., Research Triangle Park, NC). Generally, the tensile strengths of five samples are measured and averaged. Tensile strength generally has units of grams force per unit sample width, such as g/25.4 mm.

### Wet Tensile Strength

Tensile testing of wet tissue samples, such as laminates of the present invention, is conducted on a tensile testing machine maintaining a constant rate of elongation and the width of each specimen tested is one (1) inch. Generally products are tested in their product forms without separating into individual plies. For example, a 2-ply product is tested as two plies and recorded as such. The tensile tester, which is the same as described above, parameters are as follows:

| | |
|---|---|
| Crosshead | 304.8 ± 10 mm/min |
| Speed | (12 ± 0.4 inch/minute) |
| Gage | 76 ± 1 mm (3 ± 0.04 inch) |
| Length | |
| Load Units | Gram-force |
| Full-Scale | Use an appropriate load cell |
| Load | for the material being tested so |
| | the values fall between 5 and 95 |
| | percent of the full-scale load. |
| Break | 70% |
| Sensitivity | |
| Slope | 70 and 157 grams |
| Preset Points | |

Wet tensile strength measurements can be performed on pre-moistened wipes or can be performed on dry base sheets. When testing dry base sheets, prior to testing, samples are soaked in tap water at room temperature. In such instances, 5 dry sheets of a sample are weighed and combined with 220% of water based on the weight of the sheets. The water is added to the sheets so that the sheets are saturated. The water can be applied to each side of the dry sheets using a syringe. The saturated sheets are then placed in a ZYPLOK bag and stored for a week. After a week, the samples are removed, cut into 1 inch strips, and tested immediately.

Ten representative specimens are tested, and the arithmetic average of all individual specimen tests is recorded as the appropriate MD or CD tensile strength having units of grams per one inch (g/in).

### Debris

Debris is generally measured using a MasterScreen™ from Pulmac Systems International (Williston, VT). The MasterScreen™ is a low consistency screening device designed to mechanically separate fibers from contaminants. The MasterScreen™ is fitted with a screen (part no. 3390P) having a slot size of 100 μm (0.004 inches). Screening of pulps using a MasterScreen type instrument is generally described in T-274.

Approximately 5.0 bone dry grams of fiber are used for the analysis. The sample may be taken from a handsheet, a pulpsheet or from wet lap pulp. The 5.0 g sample is mixed with 2 L of water and disintegrate using a benchtop disintegrator at 15,000 Revolution prior to testing. The sample is screened according to the manufacturer's instructions and the rejects are collected in a collection cup fitted with a 150 mesh stainless steel screen. A wash cycle is run after the initial cycle to ensure that all of the debris retained by the screen is captured. Finally, the collection cup is rinsed with water and the rinse fluid is collected in a beaker. The rejects and wash fluid collected in the beaker is filtered under vacuum using a pre-weighed filter pad. Debris is collected on the filter pad, which is dried in an oven preheated to 105° C. overnight. The dried filter pad is weighed to the nearest 0.01 g and the weight percentage of debris is calculated. Generally, debris is reported as wt % and is the average of three samples.

### Dispersibility Test in Seconds (Slosh Box Test)

This method uses a bench-scaled apparatus to evaluate the breakup or dispersibility of flushable consumer products as they travel through the wastewater collection system and is based on Test IWSFG PAS 3:2018 published by the International Wastewater Services Flushability Group. In this test method, a clear plastic tank is loaded with a product and tap water or raw wastewater. The container is then moved up and down by a cam system at a specified rotational speed to simulate the movement of wastewater in the collection system. The initial breakup point and the time for dispersion of the product into pieces measuring 1 in×1 in (25 mm×25 mm) are recorded. This 1 in×1 in (25 mm×25 mm) size is a parameter that is used because it reduces the potential of product recognition. The testing can be extended until the product is fully dispersed. The various components of the product are then screened and weighed to determine the rate and level of disintegration.

### Testing Parameters:

The slosh box water transport simulator according to Test IWSFG PAS 3:2018 consists of a transparent plastic tank that is mounted on an oscillating platform with speed and holding time controller. The angle of incline produced by the cam system produces a water motion equivalent to 60 cm/s (2 ft/s), which is the minimum design standard for wastewater flow rate in an enclosed collection system. The rate of oscillation is controlled mechanically by the rotation of a cam and level system and should be measured periodically throughout the test. This cycle mimics the normal back- and forth movement of wastewater as it flows through a sewer pipe.

### Test Initiation:

Room temperature tap water (softened and/or non-softened) or raw wastewater (2000 mL) is placed in the plastic container/tank. The timer is set for six hours (or longer) and cycle speed is set for 26 rpm. The pre-weighed product is placed in the tank and observed as it undergoes the agitation period. For toilet tissue, add a number of sheets that range in weight from 1 to 3 grams. All other products may be added whole with no more than one article per test. A minimum of one gram of test product is recommended so that adequate loss measurements can be made. The time to first breakup and full dispersion are recorded. Note: For pre-moistened products it is recommended to flush them down the toilet and drain line apparatus prior to putting them into the slosh box apparatus or rinse them by some other means. Other pre-rinsing techniques should be described in the study records.

### Test Termination:

The test is terminated when the product reaches a dispersion point of no piece larger than 1 in ×1 in (25 mm×25 mm) square in size or at the designated destructive sampling points. The amount of time to reach this point is measured.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a dispersible moist wipe that not only rapidly disperses in water but also has sufficient wet tensile strength during use. In accordance with the present disclosure, the dispersible moist wipe can be produced containing non-wood pulp fibers, such as Hesperaloe fibers. In fact, the product of the present disclosure can be constructed without containing any wood fibers, such as softwood fibers. Instead, the product can be made containing primarily non-wood fibers optionally in combination with staple fibers, such as regenerated cellulose staple fibers.

The product of the present disclosure can provide various advantages and benefits. For instance, the non-wood pulp fibers present a much more environmentally friendly option in comparison to using wood fibers. The non-wood fibers also have a blend of characteristics making them particularly well suited for producing products with good wet strength in combination with low dispersibility times. Further, use of the non-wood fibers, such as Hesperaloe fibers, allows for the minimization of the use of other fibers such as staple fibers. For instance, base sheets can be made according to the present disclosure containing staple fibers in an amount of about 50% by weight or less, such as less than about 30% by weight, such as less than about 25% by weight, such as less than about 20% by weight, such as less than about 15% by weight, such as less than about 10% by weight, such as less than about 5% by weight.

As will be demonstrated in the examples below, dispersible moist wipes can be produced that have an excellent and optimum combination of wet tensile strength and dispersibility. For example, moist wipes in accordance with the present disclosure can display a wet geometric mean tensile (GMT) strength of greater than about 250 g/in, such as greater than about 300 g/in, such as greater than about 350 g/in, such as greater than about 400 g/in, such as greater than about 450 g/in, such as greater than about 500 g/in, such as greater than about 550 g/in, such as greater than about 600 g/in. The wet geometric mean tensile strength of the moist wipe can be less than about 1,400 g/in, such as less than about 1,000 g/in.

The dispersibility time of moist wipes made according to the present disclosure can be related to the wet geometric mean tensile strength. For instance, in one aspect, the dispersibility time in seconds can be less than the following relationship:
1.3×the wet GMT;

wherein wet GMT is in units of g/1″ and wet GMT can range from about 50 g/in to about 1,400 g/in. The dispersibility time can be less than about 1,000 seconds, such as less than about 800 seconds, such as less than about 600 seconds, such as less than about 400 seconds, such as less than about 350 seconds, such as less than about 300 seconds, such as less than about 250 seconds, such as less than about 200 seconds, such as less than about 180 seconds, such as less than about 150 seconds. The dispersibility time is generally greater than about 5 seconds, such as greater than about 30 seconds.

As described above, base sheets and moist wipes made according to the present disclosure are primarily made from non-wood pulp fibers. The non-wood fibers can be derived from one or more plants of the genus Hesperaloe. The Hesperaloe fibers, for instance, can be obtained from Hesperaloe funifera, Hesperaloe parviflora, Hesperaloe noctuma, Hesperaloe chiangic, Hesperaloe tenuifolia, Hesperaloe engelmannii, Hesperaloe malacophylla, or mixtures thereof. In one particular aspect, the Hesperaloe fibers comprise Hesperaloe funifera fibers.

Of particular advantage, the non-wood fibers can be pulped without using the same pulping process for wood fibers. The pulping process for the non-wood fibers in comparison can be more environmentally friendly and requires less energy. The pulping process for the non-wood fibers, for instance, can comprise a mechanical pulping process alone or in combination with chemical treatments.

In one aspect, the pulping process for the non-wood fibers or Hesperaloe fibers can include harvesting biomass and cutting the biomass to an optimum size, such as having a nominal size of less than about 20 mm, such as less than about 10 mm. The cut biomass can then be passed through a press that compresses and mechanically treats the biomass in order to remove water-soluble components. The extracted and cut biomass can then be subjected to a second compression, optionally with maceration, using a screw press while impregnating the biomass with a first alkaline peroxide solution. The impregnated bagasse can then be fed to a refiner and combined with a second alkaline peroxide solution to form a primary pulp. During refining, the biomass can be fibrillated into pulp. After refining, the primary pulp can be diluted and subjected to cleaning and/or screening to further remove debris prior to an optional bleaching process. During cleaning, epidermal debris can be removed for producing a pulp having very low debris amount. For instance, the resulting pulp can contain debris in an amount less than about 2.5% by weight, such as in an amount less than about 2% by weight, such as in an amount less than about 1.5% by weight, such as in an amount less than about 1% by weight. One embodiment of a process for pulping non-wood pulp is described in PCT Publication No. WO 2022/098963, which is incorporated herein by reference.

As described above, in producing the non-wood pulp fibers, the biomass is first cut to a desired size. The biomass may be cut to size at the time of harvesting using a forage harvester. The forage harvester, for instance, can use reciprocating knives, disc or rotary mowers, or large saw-like blades. The plant can be cut above the crown such as from about 10 to about 30 cm above the ground. The nominal chop length can be from about 5 to about 50 mm, such as from about 5 to about 30 mm, such as from about 5 to about 20 mm.

Cutting the biomass before the biomass is pulped or bleached can reduce the fraction of long fibers in the pulp making the pulp more readily dispersible and amenable for use in the manufacture of moist wiping products. Further, the reduction in long fiber fraction may be achieved without a significant reduction in the fiber length. The resulting pulp, for instance, can have a length weighted average fiber length of greater than about 1.5 mm, such as greater than about 1.75 mm, such as greater than about 1.8 mm, such as greater than about 2 mm, such as greater than about 2.2 mm, and less than about 3 mm.

After the biomass is harvested and cut, the biomass can be treated prior to pulping to remove at least a portion of the biomass epidermis and at least a portion of the water-soluble solids. Removal of the epidermis and water-soluble solids can be carried out simultaneously. The biomass epidermis originates from the cuticle of biomass leaves and is desirably removed for improving the efficiency of pulping and bleaching. In addition, removal of the epidermis reduces debris in the final product and may improve the physical properties of the resulting fibers. Removing the epidermis, for instance, can improve hand feel and softness and reduced lint in products made from the fibers.

Water-soluble solids and biomass epidermis can be removed, for instance, by subjecting the biomass to screw devices designed to compress and mechanically treat the biomass. The screw device can be a plug screw or other form of compression screw. In one aspect, the device is capable of a compression ratio of at least 2:1, such as at least about 2.5:1, such as at least about 3:1, such as from about 2:1 to about 5:1.

During compression, the biomass can also be subjected to maceration for softening and separating the biomass into fibers and removing the epidermis and water-soluble solids. Compression can take place under a pressure of at least about 0.2 bars, such as at least about 0.5 bars, such as at least about 1 bar, such as from about 0.5 bars to about 1.5 bars.

The extracted and macerated bagasse is then converted to pulp by mechanical refining with, or without, the addition of chemicals, such as alkaline-based chemicals.

In one aspect, pulping is carried out using an alkaline peroxide mechanical pulping process. During the process, hydrogen peroxide and alkali can be added to the bagasse before or during fibrillation in a refiner. For instance, the bagasse can be impregnated by first an alkaline peroxide solution in the compression and maceration device. The impregnated bagasse can then be fed to a pulping device containing a rotating disc. A second alkaline peroxide solution can be added to the impregnated bagasse as it is fed to the pulping device.

The pulping device may operate in continuous or batch mode. The pulping device may be operated at a temperature of from about 120° C. to about 190° C. The pulping device can produce a primary pulp under conditions that allow continued reaction between the alkaline peroxide chemicals and the pulp. For example, in one aspect, the pulp may be discharged to a retention vessel and retained for an hour or more at a temperature of at least about 80° F.

In one aspect, the primary pulp produced as described above can be mixed and transferred to a bleaching tower for secondary bleaching. The primary pulp can be fed to the bleaching tower at a temperature greater than about 80° C., such as from about 80° C. to about 85° C. and at a pH of greater than about 8.5, such as greater than about 9, such as greater than about 9.5. In one aspect, the primary pulp is quenched as it is fed to the bleaching tower for cooling the pulp to less than about 80° C. Bleaching can occur in the bleaching tower without the use of chlorine or chlorine-containing compounds. For instance, bleaching can be carried out using a non-chlorine oxidizing agent, such as peroxides, oxygen and/or ozone. Bleaching may be carried out at a temperature of from about 80° C. to about 85° C. and the retention time may range from about one hour to about five hours. The final pH of the bleached pulp can be from about 9 to about 11, such as from about 9 to about 10.

The resulting bleached pulp may be fed to a further processing step which can include mechanical refining, screening, washing, or the like, to produce the final pulp.

The non-wood pulp or Hesperaloe pulp can contain a relatively low degree of fines and a high freeness. The fines content, for instance, can be less than about 5% by weight, such as less than about 2% by weight, such as less than about 1% by weight. The non-wood pulp fibers can display a freeness of greater than about 400 mL, such as greater than about 450 mL, such as greater than about 500 mL, and less than about 600 mL. The non-wood pulp can have a brightness of greater than about 80%, such as greater than about 81%, such as greater than about 82%, and generally less than about 92%. As described above, the pulp can contain little to no debris. For instance, the debris content can be less than about 1% by weight, such as less than about 0.9% by weight, such as less than about 0.8% by weight.

In producing base sheets in accordance with the present disclosure, the non-wood pulp fibers or Hesperaloe pulp fibers can be fed to a process for producing nonwoven webs. The webs can be made using any suitable process. For instance, the webs can be made in a wetlaid process or a foam forming process. The non-wood pulp fibers or Hesperaloe pulp fibers can be used alone or in combination with other fibers for producing the webs. The non-wood pulp fibers or Hesperaloe fibers generally have a low coarseness. For instance, the fibers can display a coarseness of less than about 10 mg/100 m (1 dTex). For instance, the non-wood pulp fibers or Hesperaloe pulp fibers can display a coarseness of less than about 10.0 mg/100 m, such as less than about 8.0 mg/100 m, such as less than about 6.0 mg/100 m, such as from about 3.0 to about 10.0 mg/100 m, such as from about 3.5 mg/100 m to about 7.0 mg/100 m. In one aspect, the web is also subjected to a hydroentangling process for improving wet strength while retaining dispersibility.

In general, base sheets or nonwoven webs made according to the present disclosure contain the non-wood pulp fibers or Hesperaloe pulp fibers in an amount greater than 50% by weight. For instance, the base sheet can contain the non-wood pulp fibers or Hesperaloe pulp fibers in an amount greater than about 60% by weight, such as in an amount greater than about 70% by weight, such as in an amount greater than about 75% by weight, such as in an amount greater than about 80% by weight, such as in an amount greater than about 85% by weight, such as in an amount greater than about 90% by weight, such as in an amount greater than about 95% by weight. In one aspect, 100% of the fibers contained in the base sheet can be comprised of the non-wood pulp fibers or Hesperaloe pulp fibers.

Optionally, the non-wood pulp fibers or Hesperaloe pulp fibers can be combined with other fibers, such as staple fibers. In one aspect, the staple fibers may comprise regenerated cellulose fibers.

The regenerated fibers are man-made filaments obtained by extruding or otherwise treating regenerated or modified cellulosic materials from woody or non-woody plants, as is known in the art. For example, but not by way of limitation, the regenerated fibers may include one or more of lyocell, viscose, rayon, and the like. In some embodiments, the regenerated fibers have an average fiber length in the range of about 3 to about 20 millimeters, such as from about 6 to about 12 millimeters. Additionally, in some embodiments, the regenerated fibers may have a decitex in the range of about 0.7 g/10,000 m to about 2 g/10,000 m. Moreover, the decitex may be in the range of about 0.9 g/10,000 m to about 1.1 g/10,000 m. In one suitable embodiment, the regenerated fibers are not mechanically treated to alter or otherwise affect the shape the fiber. More specifically, the regenerated fibers are not fibrillated.

Of particular advantage, the amount of regenerated cellulose fibers contained in base sheets made according to the present disclosure can be minimized due to the use of the non-wood pulp fibers. For instance, base sheets can be constructed in accordance with the present disclosure that contain regenerated cellulose staple fibers in an amount no more than 50% by weight. For instance, the base sheet can contain regenerated cellulose staple fibers in an amount less than about 30% by weight, such as in an amount less than about 25% by weight, such as in an amount less than about 20% by weight, such as in an amount less than about 15% by weight, such as in an amount less than about 10% by weight, such as in an amount less than about 5% by weight.

Optionally, the staple fibers can include polymer synthetic fibers in combination with or as a substitute for the regenerated cellulose fibers. However, in certain instances, the base sheets or nonwoven webs made according to the present disclosure may be substantially free from, or free from, synthetic fibers. Hence, in one aspect, the sheet products of the present disclosure can be constructed so as not to contain any thermoplastic polymers, particularly fossil-based polymers. For example, the sheet products of the present disclosure can be free of polyolefin polymers (e.g. fibers) and/or free of polyester polymers (e.g. fibers).

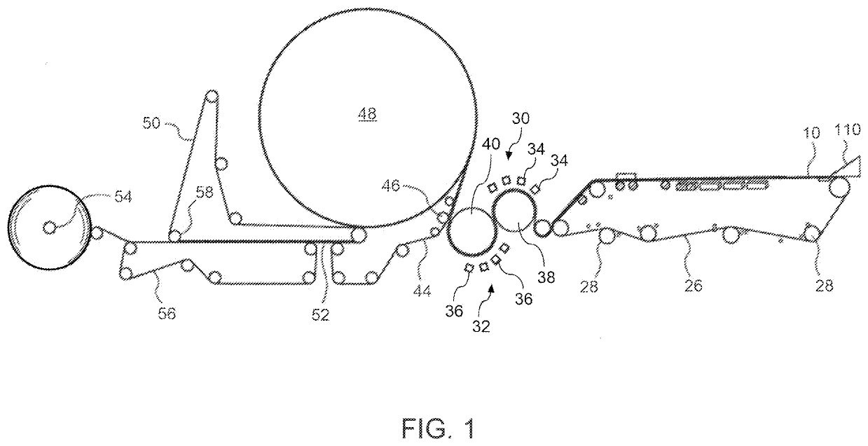

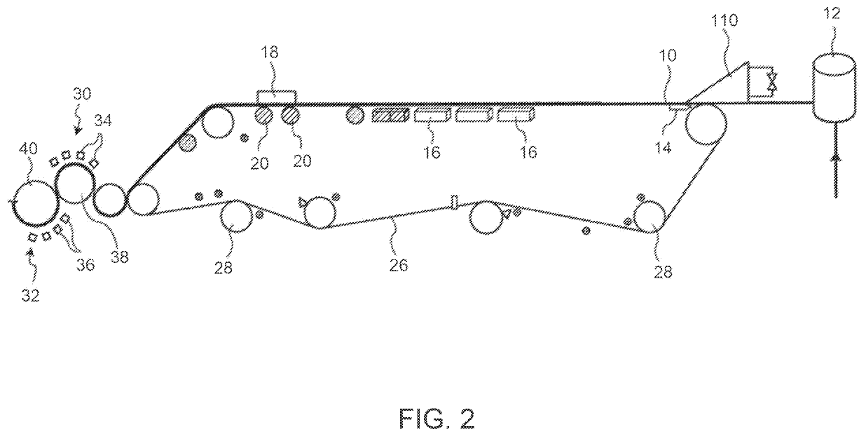

As described above, base sheets or nonwoven webs made according to the present disclosure can use any suitable process. The process, for instance, can be a wetlaid process incorporating hydroentangling jets or can be a foam forming process incorporating hydroentangling jets. In one aspect, for instance, a foam forming process is used that is particularly well suited to accommodating longer fibers. FIGS. 1 and 2, for instance, represent one embodiment of a foam forming process that may be used to produce base sheets in accordance with the present disclosure. It should be understood, however, that the embodiment illustrated in FIGS. 1 and 2 is merely for exemplary purposes.

In one aspect, the process includes first selecting a fiber furnish containing primarily non-wood pulp fibers, particularly Hesperaloe pulp fibers. The fiber furnish is then fed to a web forming process which can be a foam forming process in which the newly formed web is also subjected to a hydroentangling step. After hydroentangling the fibers, the nonwoven web can then be fed to a drying process. The drying process can include through-air dryers, heated drums, or combinations thereof.

When the base sheet is foam formed, the fiber furnish can be combined with a foam to create a foamed suspension. The fibers, for instance, can be blended with water and a foaming agent.

The foaming agent, for instance, may comprise any suitable surfactant. In one embodiment, for instance, the foaming agent may comprise sodium lauryl sulfate, which is also known as sodium laureth sulfate or sodium lauryl ether sulfate. In one embodiment, the foaming agent is a nonionic surfactant which may comprise an alkyl polyglycoside. The foaming agent, for instance, can be a C8 alkyl polyglycoside, a C10 alkyl polyglycoside, or a mixture of C8 and C10 alkyl polyglycosides.

Other foaming agents include sodium dodecyl sulfate or ammonium lauryl sulfate. In other embodiments, the foaming agent may comprise any suitable cationic and/or amphoteric surfactant. For instance, other foaming agents include fatty acid amines, amides, amine oxides, fatty acid quaternary compounds, and the like.

The foaming agent is combined with water generally in an amount greater than about 0.1% by weight, such as in an amount greater than about 1% by weight, such as in an amount greater than about 2% by weight, such as in an amount greater than about 3% by weight. One or more foaming agents are generally present in an amount less than about 50% by weight, such as in an amount less than about 10% by weight, such as in an amount less than about 8% by weight, such as in an amount less than about 4% by weight.

Once the foaming agent and water are combined, the mixture is blended or otherwise subjected to forces capable of forming a foam. A foam generally refers to a porous matrix, which is an aggregate of hollow cells or bubbles which may be interconnected to form channels or capillaries.

The foam density can vary depending upon the particular application and various factors including the fiber furnish used. In one embodiment, for instance, the foam density of the foam can be greater than about 200 g/L, such as greater than about 250 g/L, such as greater than about 300 g/L. The foam density is generally less than about 600 g/L, such as less than about 500 g/L, such as less than about 400 g/L, such as less than about 350 g/L. In one embodiment, for instance, a lower density foam is used having a foam density of generally less than about 350 g/L, such as less than about 340 g/L, such as less than about 330 g/L. The foam will generally have an air content of greater than about 30%, such as greater than about 40%, such as greater than about 50%, such as greater than about 60%. The air content is generally less than about 80% by volume, such as less than about 70% by volume, such as less than about 65% by volume.

In order to form the nonwoven web, the foam is combined with a selected fiber furnish in conjunction with any auxiliary agents. The foamed suspension of fibers is then pumped to a tank and from the tank is fed to a headbox. FIGS. 1 and 2, for instance, show one embodiment of a process in accordance with the present disclosure for forming the web. As shown particularly in FIG. 2, the foamed fiber suspension can be fed to a tank 12 and then fed to the headbox 110. From the headbox 110, the foamed fiber suspension is issued onto an endless traveling forming fabric 26 supported and driven by rolls 28 in order to form a web 10. As shown in FIG. 2, a forming board 14 may be positioned below the web 10 adjacent to the headbox 110. Once formed on the forming fabric 26, the foam formed web can have a consistency of less than about 50%, such as less than about 20%, such as less than about 10%, such as less than about 5%. In fact, the forming consistency can be less than about 2, such as less than about 1.8, such as less than about 1.5. The forming consistency is generally greater than about 0.5, such as greater than about 0.8. The forming consistency indicates the ability to produce webs according to the present disclosure while minimizing the amount of water needed during formation.

Once the wet web is formed on the forming fabric 26, the web is conveyed downstream and dewatered. For instance, the process can optionally include a plurality of vacuum devices 16, such as vacuum boxes and vacuum rolls. The vacuum boxes assist in removing moisture from the newly formed web 10.

As shown in FIG. 2, the forming fabric 26 may also be placed in communication with a steambox 18 positioned above a pair of vacuum rolls 20. The steambox 18, for instance, can increase dryness and reduce cross-directional moisture variance. The applied steam from the steambox 18 heats the moisture in the wet web 10 causing the water in the web to drain more readily, especially in conjunction with the vacuum rolls 20. From the forming fabric 26, the newly formed web 10, in the embodiment shown in FIG. 1, is conveyed downstream, subjected to hydroentangling, and dried on a through-air dryer.

After the foam formed web has been produced, the web is subjected to one or more hydroentangling steps. In the embodiment illustrated in FIG. 2, for instance, the web 10 is subjected to two different hydroentangling steps. In particular, in FIG. 2, the web 10 is hydroentangled on a first surface during a first hydroentangling step and then hydroentangled on a second and opposite surface during a second hydroentangling step. As shown in FIG. 2, for example, the process can include a first hydroentangling device 30 and a second hydroentangling device 32. The hydroentangling that occurs at each hydroentangling station may be accomplished utilizing conventional hydroentangling equipment. The hydroentangling of the foam formed web may be carried out with any appropriate working fluid such as, for example, water. The working fluid flows through a manifold which evenly distributes the fluid through a series of individual holes or orifices. Exemplary holes or orifices, for example, can have a diameter of from about 10 microns to about 200 microns. For example, the manifold may include a strip of orifices having a diameter of about 20 to about 50 microns. The manifold may contain about 20 to about 40 holes per inch and can include 1 to 3 rows of holes. Many other manifold configurations and combinations may be used. In the embodiment illustrated in FIG. 2, for instance, the hydroentangling device 30 includes a plurality of injectors 34, while the hydroentangling device 32 includes a plurality of injectors 36. The injectors 34 and 36 can be part of the manifold and can be in communication with a working fluid supply. In the embodiment illustrated in FIGS. 1 and 2, the first hydroentangling device 30 includes four banks of water jets or injectors 34 and the second hydroentangling device 32 also includes four banks of water jets or injectors 36. It should be understood, however, that each hydroentangling device can include a single bank, two banks, three banks, four banks, five banks, six banks, or more of water jets.

During the hydroentangling process, the working fluid can pass through the orifices at pressures ranging from about 10 bar to about 300 bar, such as from about 20 bar to about 250 bar. Suitably, no binders are used to supplement or otherwise increase the bonds between the fibers in the nonwoven web being formed. Rather, the bonds between the fibers of the nonwoven web are created through hydroentangling. Thus, hydroentangling can increase the strength, particularly the wet strength of the nonwoven web or base sheet. In order to preserve dispersibility, relatively low pressures can be used when hydroentangling the web. For instance, the fluid pressure can be less than about 100 bar.

The fluid impacts the material or web which can be supported on a foraminous surface or wire or may be supported on a porous drum surface. In the embodiment illustrated in FIG. 2, for instance, hydroentangling occurs on a first drum 38 and a second drum 40.

When supported on a foraminous surface or wire during hydroentangling, the wire can have a mesh size of from about 40×40 to about 100×100. The wire or surface may also be a multi-ply mesh having a mesh size of from about 50×50 to about 200×200. In one aspect, only one side of the web 10 is hydroentangled.

As described above, alternatively, the web 10 can be placed directly onto the surface of the drum 38 and on the surface of the drum 40 during hydroentangling. Each drum can include a plurality of openings or vacuum passages for withdrawing excess water. These openings or vacuum passages can also create a pattern into the web 10 during the hydroentangling process. For example, a pattern can be formed into one surface of the web at the first hydroentangling station and a pattern can be formed into the second and opposite surface of the web at the second hydroentangling station. The pattern formed into each surface of the web 10 can be highly distinctive and can increase the aesthetic appeal of nonwoven materials made from the web. In addition, the pattern formed into the web can be three-dimensional including hills and valleys. This three-dimensional topography can further improve various properties of the material.

Once the foam formed web 10 is hydroentangled one or more times, the web can be dried using a non-compressive drying operation. For example, as shown in FIG. 1, the foam formed web can be dried using a through-air dryer.

Referring to FIG. 1, the foam formed and hydraulically entangled web 10 is transferred from the drum 40 to a throughdrying fabric 44 with the aid of a vacuum transfer roll 46 or a vacuum transfer shoe. If desired, the throughdrying fabric can be run at a slower speed than the web 10 to further enhance stretch. Transfer can be carried out with vacuum assistance to ensure deformation of the sheet to conform to the throughdrying fabric, thus yielding desired bulk and appearance if desired. In the embodiment illustrated in FIG. 1, the foam formed web 10 is transferred to a throughdrying fabric 44. Alternatively, the foam formed web can be transferred to a metal, porous sleeve that forms the circumference of the throughdryer 48.

Alternatively, the foam formed web 10 can be conveyed on the throughdrying fabric 44 over the circumference of the throughdryer 48. The throughdrying fabric can contain high and long impression knuckles. For example, the throughdrying fabric can have about from about 5 to about 300 impression knuckles per square inch which are raised at least about 0.005 inches above the plane of the fabric. During drying, the web can be further macroscopically arranged to conform to the surface of the throughdrying fabric. Flat surfaces, however, can also be used in the present disclosure.

The side of the web contacting the throughdrying fabric is typically referred to as the “fabric side” of the nonwoven web. The fabric side of the nonwoven web, as described above, may have a shape that conforms to the surface of the throughdrying fabric after the fabric is dried in the throughdryer. The opposite side of the nonwoven web, on the other hand, is typically referred to as the “air side”. The air side of the web is typically smoother than the fabric side during normal throughdrying processes.

The level of vacuum used for the web transfers can be from about 3 to about 15 inches of mercury (75 to about 380 millimeters of mercury), preferably about 5 inches (125 millimeters) of mercury. The vacuum shoe or roll (negative pressure) can be supplemented or replaced by the use of positive pressure from the opposite side of the web to blow the web onto the next fabric in addition to or as a replacement for sucking it onto the next fabric with vacuum.

The web is finally dried to a consistency of about 94 percent or greater by the throughdryer 48 and thereafter transferred to a carrier fabric 50. The dried basesheet 52 is transported to the reel 54 using carrier fabric 50 and an optional carrier fabric 56. An optional pressurized turning roll 58 can be used to facilitate transfer of the web from carrier fabric 50 to fabric 56. Suitable carrier fabrics for this purpose are Albany International 84M or 94M and Asten 959 or 937, all of which are relatively smooth fabrics having a fine pattern. Although not shown, reel calendering or subsequent off-line calendering can be used to improve the smoothness and softness of the basesheet.

In one embodiment, the resulting foam formed web 52 is a textured web, which has been dried in a three-dimensional state. The texture in the web can be created due to the hydroentangling stations, due to the manner in which the web is dried using the through-dryer 48 or can be a result of both processes. For example, the web 52 can be dried while still including a pattern formed into the web.

The basis weight of webs made in accordance with the present disclosure can vary depending upon the final product. In general, the basis weight of the products may vary from about 50 gsm to about 150 gsm. The basis weight, for instance, can be greater than about 55 gsm, such as greater than about 60 gsm, such as greater than about 65 gsm, and generally less than about 120 gsm, such as less than about 100 gsm, such as less than about 90 gsm, such as less than about 80 gsm.

The process of the present disclosure can also produce webs with good bulk characteristics. The dry bulk, for instance, can generally be greater than about 3 cc/g, such as greater than about 5 cc/g, such as greater than about 8 cc/g, and generally less than about 20 cc/g, such as less than about 15 cc/g.

In one embodiment, the nonwoven material can be cut into individual sheets. The individual sheets may have any suitable size and shape. In one embodiment, the individual sheets r can have a width of from about 8 cm to about 100 cm, such as from about 10 cm to about 50 cm, such as from about 20 cm to about 25 cm. The length of the individual sheets can be from about 10 cm to about 200 cm, such as from about 20 cm to about 100 cm, such as from about 35 cm to about 45 cm.

The individual sheets are also pre-moistened with a wetting solution, such as water, a solvent, a waterless hand cleanser, or any other suitable liquid. The liquid may contain antiseptics, surfactants, emollients, humectants, and so forth. Generally, each individual sheet contains greater than about 100 wt. %, in some embodiments from about 150 to about 1500 wt. %, and in some embodiments, from about 300 to about 1200 wt. % of the liquid based on the dry weight of the sheet. In certain embodiments the wetting solution may comprise from about 98% water to about 99.9% water, alternatively from about 98.5% water to about 99.7% water, alternatively from about 98.5% water to about 99.5% water, alternatively from about 99% water to about 99.3% water, by weight of the lotion. In certain instances the wetting solution may further comprise a preservative system for preventing microbial growth, such as an organic acid and optionally a chelating agent, such as ethylenediamine tetraacetic acid (EDTA) or a preservative enhancing agent such as sorbitan caprylate, glyceryl caprylate/caprate, or combinations thereof. Suitable organic acids include benzoic acid. Salts of organic acids may also be present, such as sodium benzoate, sodium gluconate, sodium levulinate, sodium anisate, and potassium sorbate. The wetting solution may also comprise a pH buffering system, such as a citrate-citric acid buffering system, at a pH of less than 5, such as from about 3.5 to about 5, such as from 3.6 to 4.2.

The individual sheets may be packaged in a variety of forms, materials and/or containers, including, but not limited to, rolls, boxes, tubs, flexible packaging materials, and so forth. Some examples of suitable containers include rigid tubs, film pouches, etc.

The present disclosure may be better understood with reference to the following example.

### Example

Various foam formed base sheets were constructed containing either Hesperaloe pulp fibers in combination with regenerated cellulose fibers or Northern softwood kraft fibers in combination with regenerated cellulose fibers. The foam formed base sheets were hydroentangled on one side using either three banks of injectors or four banks of injectors. The fluid pressure was between 10 bar and 60 bar. The basis weight of the samples was about 60 gsm.

After the base sheets were constructed, the base sheets were impregnated with a wetting solution comprising water and tested for wet geometric mean tensile strength and dispersibility time.

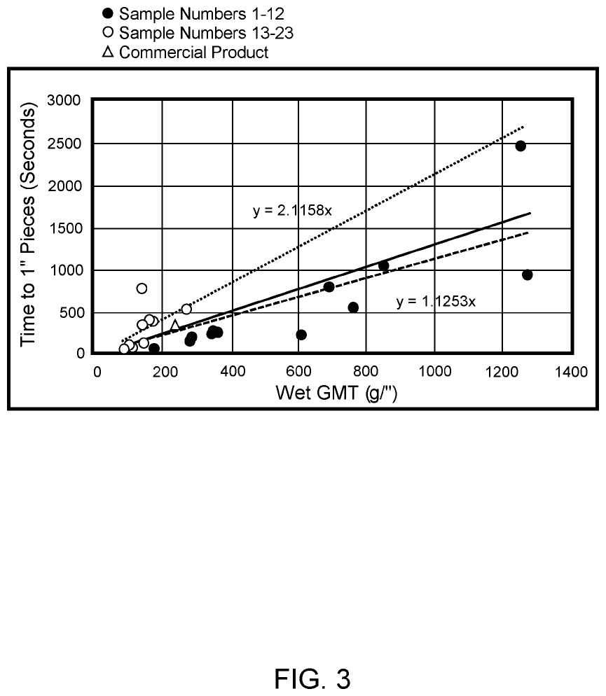

The results are presented in the table below and illustrated graphically in FIG. 3.

The Hesperaloe pulp fibers used had a freeness of 538 mL, had a length weight average fiber length of about 2.45 mm, had an arithmetic fiber length of about 1.4 mm, displayed a coarseness of about 5.2 mg/100 m, and contained fines in an amount of about 2.8%. The following results were obtained:

| | | | | Softwood | Hesperaloe | | | |
|---|---|---|---|---|---|---|---|---|
| | | | Rayon | Fibers | Fibers | No. of | Wet | |
| Sample | Natural | % | Length | Amount | Amount | Fluid | GMT | Dispersibility |
| No. | Fiber | Rayon | mm | (%) | (%) | Injectors | (g/in) | (seconds) |
| 1 | Hesperaloe | 20 | 6 | 0 | 80 | 3 | 168 | 57 |
| | fibers | | | | | | | |
| 2 | Hesperaloe | 30 | 6 | 0 | 70 | 4 | 277 | 139 |
| | fibers | | | | | | | |
| 3 | Hesperaloe | 20 | 6 | 0 | 80 | 4 | 281 | 202 |
| | fibers | | | | | | | |
| 4 | Hesperaloe | 20 | 10 | 0 | 80 | 4 | 605 | 213 |
| | fibers | | | | | | | |
| 5 | Hesperaloe | 10 | 10 | 0 | 90 | 4 | 355 | 253 |
| | fibers | | | | | | | |
| 6 | Hesperaloe | 30 | 6 | 0 | 70 | 3 | 337 | 237 |
| | fibers | | | | | | | |
| 7 | Hesperaloe | 20 | 6 | 0 | 80 | 3 | 339 | 270 |
| | fibers | | | | | | | |
| 8 | Hesperaloe | 30 | 6 | 0 | 70 | 4 | 762 | 557 |
| | fibers | | | | | | | |
| 9 | Hesperaloe | 20 | 6 | 0 | 80 | 4 | 689 | 788 |
| | fibers | | | | | | | |
| 10 | Hesperaloe | 20 | 10 | 0 | 80 | 4 | 1277 | 936 |
| | fibers | | | | | | | |
| 11 | Hesperaloe | 10 | 10 | 0 | 90 | 4 | 852 | 1048 |
| | fibers | | | | | | | |
| 12 | Hesperaloe | 20 | 6 | 0 | 80 | 4 | 1250 | 2452 |
| | fibers | | | | | | | |
| 13 | Softwood | 20 | 6 | 80 | 0 | 3 | 79 | 36 |
| | fibers | | | | | | | |
| 14 | Softwood | 20 | 6 | 80 | 0 | 4 | 85 | 42 |
| | fibers | | | | | | | |
| 15 | Softwood | 30 | 6 | 70 | 0 | 4 | 92 | 45 |
| | fibers | | | | | | | |
| 16 | Softwood | 20 | 6 | 80 | 0 | 3 | 94 | 98 |
| | fibers | | | | | | | |
| 17 | Softwood | 30 | 6 | 70 | 0 | 3 | 105 | 76 |
| | fibers | | | | | | | |
| 18 | Softwood | 20 | 6 | 80 | 0 | 4 | 133 | 339 |
| | fibers | | | | | | | |
| 19 | Softwood | 30 | 6 | 70 | 0 | 4 | 154 | 406 |
| | fibers | | | | | | | |
| 20 | Softwood | 10 | 10 | 90 | 0 | 4 | 165 | 377 |
| | fibers | | | | | | | |
| 21 | Softwood | 20 | 10 | 80 | 0 | 4 | 136 | 125 |
| | fibers | | | | | | | |
| 22 | Softwood | 20 | 10 | 80 | 0 | 4 | 266 | 528 |
| | fibers | | | | | | | |
| 23 | Softwood | 20 | 6 | 80 | 0 | 3 | 135 | 786 |
| | fibers | | | | | | | |

A commercial hydroentangled product was also tested that comprised a spunlace product containing wood pulp fibers in combination with regenerated cellulose fibers. The commercial product displayed a geometric mean tensile strength of 231 g/in and a dispersibility time of 320 seconds. The results are illustrated in FIG. 3. As shown, samples made according to the present disclosure displayed a dispersibility time of less than about 1.3×the wet geometric mean tensile strength. Consequently, the products made according to the present disclosure generally displayed a greater wet strength in combination with a lower dispersibility time in comparison to the samples made with Northern softwood kraft fibers.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A dispersible moist wipe comprising:
a base sheet containing non-wood pulp fibers in an amount of greater than 50% by weight, the non-wood pulp fibers having a coarseness of less than about 10 mg/100 m and having an average fiber length of from about 1.5 mm to about 3 mm;
a wetting solution incorporated into the base sheet; and
wherein the moist wipe has a wet geometric mean tensile strength of from about 250 g/in to about 1,400 g/in and a dispersibility time in seconds of less than 1.3× the wet geometric mean tensile strength of the moist wipe.

2. A dispersible moist wipe as defined in claim 1, wherein the moist wipe displays a wet geometric mean tensile strength of greater than about 350 g/in.

3. A dispersible moist wipe as defined in claim 1, wherein the moist wipe displays a wet geometric mean tensile strength of greater than about 550 g/in.

4. A dispersible moist wipe as defined in claim 1, wherein the moist wipe displays a dispersibility time of less than about 500 seconds.

5. A dispersible moist wipe as defined in claim 1, wherein the non-wood fibers are derived from one or more plants of the genus Hesperaloe.

6. A dispersible moist wipe as defined in claim 5, wherein the non-wood fibers are obtained from Hesperaloe funifera, Hesperaloe parviflora, Hesperaloe noctuma, Hesperaloe chiangic, Hesperaloe tenuifolia, Hesperaloe engelmannii, Hesperaloe malacophylla, or mixtures thereof.

7. A dispersible moist wipe as defined in claim 6, wherein the non-wood fibers comprise Hesperaloe funifera fibers.

8. A dispersible moist wipe as defined in claim 1, wherein the non-wood fibers comprise bleached non-wood fibers.

9. A dispersible moist wipe as defined in claim 1, wherein the non-wood fibers are contained in the base sheet in an amount greater than about 60% by weight.

10. A dispersible moist wipe as defined in claim 1, wherein the base sheet further contains staple fibers.

11. A dispersible moist wipe as defined in claim 10, wherein the staple fibers comprise regenerated cellulose fibers, the regenerated cellulose fibers having an average fiber length of from about 4 mm to about 14 mm.

12. A dispersible moist wipe as defined in claim 10, wherein the staple fibers have a decitex of from about 0.7 g/10,000 m to about 2 g/10,000 m.

13. A dispersible moist wipe as defined in claim 1, wherein the non-wood fibers display a freeness of from about 400 mL to about 600 mL.

14. A dispersible moist wipe as defined in claim 1, wherein the base sheet has been hydroentangled.

15. A dispersible moist wipe as defined in claim 1, wherein the base sheet has been foam formed.

16. A dispersible moist wipe as defined in claim 1, wherein the base sheet comprises a single ply web that is non-layered.

17. A dispersible moist wipe as defined in claim 1, wherein the base sheet has a basis weight of from about 50 gsm to about 150 gsm.

18. A dispersible moist wipe as defined in claim 1, wherein the only fibers contained in the base sheet are the non-wood fibers optionally combined with regenerated cellulose staple fibers.

19. A wiping product comprising a plurality of individual sheets of disposable moist wipes as defined in claim 1, wherein the plurality of individual sheets are in a stacked arrangement.

20. A dispersible moist wipe comprising:
a base sheet containing Hesperaloe funifera pulp fibers in an amount of greater than about 70% by weight, the Hesperaloe funifera pulp fibers having an average fiber length of from about 1.5 mm to about 3 mm, and wherein the base sheet has been hydroentangled;
a wetting solution incorporated into the base sheet; and
wherein the moist wipe has a wet geometric mean tensile strength of from about 250 g/in to about 1,400 g/in and a dispersibility time in seconds of less than 1.3× the wet geometric mean tensile strength of the moist wipe.

21. A method of making a dispersible moist wipe comprising:
forming a fibrous suspension, the fibrous suspension containing non-wood pulp fibers in an amount of greater than 50% by weight based on the weight of fibers present, the non-wood pulp fibers having a coarseness of less than about 10 mg/100 m and having an average fiber length of from about 1.5 mm to about 3 mm, the suspension of fibers optionally containing staple fibers, the staple fibers comprising regenerated cellulose fibers, the fibers being suspended within a foam;
depositing the foamed suspension of fibers onto a forming surface to form a base sheet;
subjecting the base sheet to a plurality of liquid jets in order to hydroentangle the fibers;
drying the base sheet; and
incorporating into the base sheet a wetting solution for forming a dispersible moist wipe, the moist wipe having a wet geometric mean tensile (GMT) strength of from about 250 g/in to about 1,400 g/in and a dispersibility time in seconds of less than 1.3× the wet geometric mean tensile strength of the moist wipe.
